SEQUENCE LISTING (17 sequences)

SEQ ID NO: 1 - protein, 8 aa, Artificial Sequence
  Description of Artificial Sequence Synthetic peptide
QILSKLRL

SEQ ID NO: 2 - protein, 3 aa, Artificial Sequence
  Description of Artificial Sequence Synthetic peptide
QIL

SEQ ID NO: 3 - protein, 3 aa, Artificial Sequence
  Description of Artificial Sequence Synthetic peptide
ILS

SEQ ID NO: 4 - protein, 3 aa, Artificial Sequence
  Description of Artificial Sequence Synthetic peptide
LSK

SEQ ID NO: 5 - protein, 3 aa, Artificial Sequence
  Description of Artificial Sequence Synthetic peptide
SKL

SEQ ID NO: 6 - protein, 3 aa, Artificial Sequence
  Description of Artificial Sequence Synthetic peptide
KLR

SEQ ID NO: 7 - protein, 3 aa, Artificial Sequence
  Description of Artificial Sequence Synthetic peptide
LRL

SEQ ID NO: 8 - protein, 4 aa, Artificial Sequence
  Description of Artificial Sequence Synthetic peptide
QILS

SEQ ID NO: 9 - protein, 4 aa, Artificial Sequence
  Description of Artificial Sequence Synthetic peptide
ILSK

SEQ ID NO: 10 - protein, 4 aa, Artificial Sequence
  Description of Artificial Sequence Synthetic peptide
LSKL

SEQ ID NO: 11 - protein, 4 aa, Artificial Sequence
  Description of Artificial Sequence Synthetic peptide
SKLR

SEQ ID NO: 12 - protein, 4 aa, Artificial Sequence
  Description of Artificial Sequence Synthetic peptide
KLRL

SEQ ID NO: 13 - protein, 5 aa, Artificial Sequence
  Description of Artificial Sequence Synthetic peptide
QILSK

SEQ ID NO: 14 - protein, 5 aa, Artificial Sequence
  Description of Artificial Sequence Synthetic peptide
ILSKL

SEQ ID NO: 15 - protein, 5 aa, Artificial Sequence
  Description of Artificial Sequence Synthetic peptide
LSKLR

SEQ ID NO: 16 - protein, 5 aa, Artificial Sequence
  Description of Artificial Sequence Synthetic peptide
SKLRL

SEQ ID NO: 17 - protein, 8 aa, Artificial Sequence
  Description of Artificial Sequence Synthetic peptide
  MOD_RES (1)..(8): D-amino acid
QILSKLRL

(19) United States
(12) Patent Application Publication
(10) Pub. No.: US 2017/0281508 A1
(43) Pub. Date: Oct. 5, 2017

(54) Peptides and Their Use in the Treatment of Skin

(71) Applicant: AVON PRODUCTS, INC., New York, NY (US)

(72) Inventors: Jolanta Idkowiak-Baldys, Montebello, NY (US); John W. Lyga, Basking Ridge, NJ (US); Uma Santhanam, Tenafly, NJ (US)

(21) Appl. No.: 14/767,451

(22) PCT Filed: Dec. 23, 2014
(86) PCT No.: PCT/US14/72060

§ 371 Date: Aug. 12, 2015

(51) Int. Cl. A61K 8/64; A61K 8/06; A61K 8/34; A61Q 19/08

(52) U.S. Cl. CPC A61K 8/64; A61Q 19/08; A61K 8/06; A61K 8/342

(57) ABSTRACT

Provided are methods of diminishing the signs of aging and/or improving the health of human integuments, such as skin, and compositions comprising peptides useful therefor. The compositions according to the invention comprise a peptide comprising the sequence QILSKLRL (SEQ ID NO: 1) or a derivative, or a 3-5 amino acid fragment thereof, in a topically acceptable vehicle.

### SEQUENCE LISTING

The instant application contains a Sequence Listing which has been submitted electronically in ASCII format and is hereby incorporated by reference in its entirety. Said ASCII copy, created on Dec. 19, 2014, is named SC181U-WO_SL.txt and is 3,473 bytes in size.

### FIELD OF INVENTION

The present invention relates generally to topical formulations comprising peptides and associated methods of diminishing the dermatological signs of aging and/or improving health or appearance of human skin. In particular, the invention relates to the use of a peptide comprising the sequence QILSKLRL (SEQ ID NO: 1), QIL (SEQ ID NO: 2), ILS (SEQ ID NO: 3), LSK (SEQ ID NO: 4), SKL (SEQ ID NO: 5), KLR (SEQ ID NO: 6), LRL (SEQ ID NO: 7), QILS (SEQ ID NO: 8), ILSK (SEQ ID NO: 9), LSKL (SEQ ID NO: 10), SKLR (SEQ ID NO: 11), KLRL (SEQ ID NO: 12), QILSK (SEQ ID NO: 13), ILSKL (SEQ ID NO: 14), LSKLR (SEQ ID NO: 15), or SKLRL (SEQ ID NO: 16) in topical formulations. The formulations may promote the production of collagen when topically applied to human skin and improve the appearance or health of the skin.

### BACKGROUND

Collagen is the body's major structural protein. It is composed of three protein chains wound together in a tight triple helix to form fibrils. The fibrils are cross-linked in the extracellular matrix to provide the structural scaffolding surrounding cells that helps to support cell shape and differentiation. The mesh-like collagen network binds cells together and provides the supportive framework or environment in which cells develop and function. The stimulation of collagen gives the skin its strength, durability, and smooth, plump appearance.

There is a need for agents that stimulate collagen production in human skin. It is therefore an object of the invention to provide new actives, cosmetic formulations, and methods for stimulating collagen production in human skin. It is a further object of the invention to provide methods for diminishing the signs of aging of skin, including treating, reversing, reducing, forestalling and/or preventing signs of aging, such as skin wrinkles and fine lines, sagging skin, and/or thinning skin by stimulating collagen production.

The foregoing discussion is presented solely to provide a better understanding of the nature of the problems confronting the art and should not be construed in any way as an admission as to prior art.

### SUMMARY OF THE INVENTION

In accordance with the foregoing objectives and others, the present invention provides peptides and cosmetic formulations containing them useful for improving the health and/or appearance of human integuments (skin, lips, nails, hair, etc.), particularly skin, affected by dermatological signs of photo- and intrinsic aging. The peptides of the invention are believed to be capable of increasing collagen production in skin and therefore are expected to have a beneficial effect on improving the appearance of signs of skin aging (e.g., diminishing the appearance of wrinkles and/or fine lines, tightening sagging skin, thickening thinning skin, evening skin tone, etc.).

In one aspect of the invention, peptides are provided comprising the sequence QILSKLRL (Gln-Ile-Leu-Ser-Lys-Leu-Arg-Leu) (SEQ ID NO: 1) or a derivative or fragment thereof, including, without limitation, the peptide having the structure of formula (I):

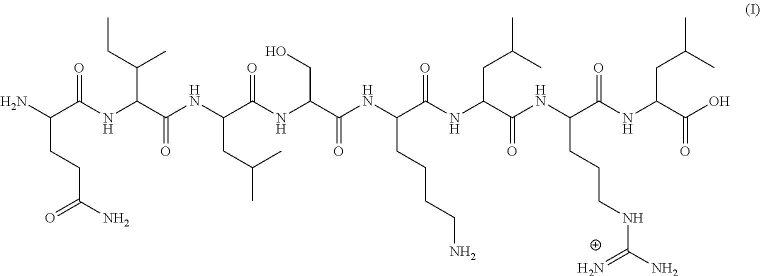 [SMILES: CCC(C)C(NC(=O)C(N)CCC(N)=O)C(=O)NC(CC(C)C)C(=O)NC(CO)C(=O)NC(CCCCN)C(=O)NC(CC(C)C)C(=O)NC(CCCNC(=N)N)C(=O)NC(CC(C)C)C(=O)O]

including zwitterions, salts, and derivatives thereof. In some implementations, each of the amino acids in the sequence QILSKLRL (SEQ ID NO: 1) is in the L optical configuration, although all stereoconfigurations are included within the scope of the invention. In some implementations, fragments of the peptide of formula (I), including three and four amino acid fragments thereof, are provided.

In another aspect of the invention compositions for topical use are provided comprising a peptide comprising the sequence QILSKLRL (SEQ ID NO: 1) or a derivative or fragment thereof, the peptide being dispersed or dissolved in a physiologically acceptable carrier or vehicle. The peptide will typically comprise from about 0.000001% to about 5% by weight of the composition, more typically, from about 0.00001% to about 2.5% by weight, or from about 0.00001% to about 1% by weight. The carrier or vehicle may comprise, for example an aqueous serum, or a water-in-oil or oil-in-water emulsion, which may further include various adjuvants such as thickeners, emulsifiers, gellants, emollients, humectants, UV absorbers, antioxidants, pH adjusters, chelators, film formers, preservatives, colorants, fragrances, and the like. The adjuvants may comprise, individually or collectively, from about 0.00001% to about 98% by weight of the composition. The topical preparations of the invention may further include one or more additional skincare active agents, such as a retinoid (e.g., retinol, retinyl palmitate, retinyl acetate, retinaldehyde, retinoic acid, etc.), an antioxidant (e.g., ascorbic acid, thiodipropionic acid or esters thereof, including dilauryl thiodipropionate), α-hydroxy acids (e.g., glycolic acid), collagenase inhibitor, anti-inflammatories, anti-acne agents, salicylic acid and derivatives, depigmenting agent, N-acetyl tyrosinamide, phytol, and botanicals, to name a few. Such additional actives may individually or collectively comprise from about 0.0001% to about 20% by weight of the composition.

In another aspect of the invention, methods for improving the appearance of human skin affected by dermatological signs of photo- and intrinsic aging and/or improving the health of human skin are provided, comprising topically applying to an area of skin in need thereof a composition comprising, in a topically acceptable vehicle, an effective amount of a peptide comprising the sequence QILSKLRL (SEQ ID NO: 1) or derivative or fragment thereof. In one implementation, the treatment of wrinkles and/or fine lines on human skin (typically, skin of the face) is provided comprising topically applying to an area of the skin in need thereof (e.g., applying to a wrinkle or fine line) a composition comprising a peptide comprising the sequence QILSKLRL (SEQ ID NO: 1) or derivative or fragment thereof, for a time sufficient to improve the aesthetic appearance of said human skin (e.g., to reduce the number or severity of wrinkles and/or fine lines). In another implementation, the treatment of sagging skin (typically, skin of the face) is provided comprising topically applying to an area of the skin in need thereof (e.g., applying to an area of sagging skin such as the cheeks or jowls) a composition comprising a peptide comprising the sequence QILSKLRL (SEQ ID NO: 1) or derivative or fragment thereof, for a time sufficient to improve the aesthetic appearance of said human skin (e.g., to tighten the sagging skin including prematurely thinned skin). In yet another implementation, the treatment of thin skin (typically, skin of the face) is provided comprising topically applying to an area of the skin in need thereof (e.g., applying to an area of thin skin) a composition comprising a peptide comprising the sequence QILSKLRL (SEQ ID NO: 1) or derivative or fragment thereof, for a time sufficient to improve the aesthetic appearance of said human skin (e.g., to thicken the skin). The treatment may be a least once or twice daily and may be continued for a period of at least four weeks, typically at least eight weeks or longer until a visible improvement is seen.

These and other aspects of the present invention will become apparent to those skilled in the art after a reading of the following detailed description of the invention, including the illustrative embodiments and examples.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

All percentages given herein refer to the weight percentages of a particular component relative to the entire composition, including the vehicle, unless otherwise indicated. It will be understood that the sum of all weight % of individual components within a composition will not exceed 100%.

All terms used herein are intended to have their ordinary meaning unless otherwise provided. The phrases “physiologically acceptable,” “topically acceptable” and “dermatologically acceptable” are used interchangeably and are intended to mean that a particular component is generally regarding as safe and non-toxic for application to a human integument (e.g., skin) at the levels employed. The term “prevent,” as used herein, includes delaying, slowing or forestalling the onset of or progression of a particular sign of skin aging. The phrase “individual in need thereof” refers to a human that could benefit from improved dermal appearance or health, including males or females. In some embodiments, the individual in need thereof is a female. The term “skin” includes, without limitation, the lips, skin of the face, hands, arms, neck, scalp, and chest. The term “thin” skin includes skin that is prematurely thinned, and may be diagnosed as such by a dermatologist. In some embodiments, the thin skin is skin of a female under the age of 40 or skin of a pre-menopausal female. As used herein, the term “consisting essentially of” is intended to limit the invention to the specified materials or steps and those that do not materially affect the basic and novel characteristics of the claimed invention, as understood from a reading of this specification.

As used herein, all terms are intended to have their ordinary meaning in the art unless specifically defined. The term “amino acid” is intended to include naturally occurring amino acids as well as non-naturally occurring amino acids and includes any small molecule (MW <1,000 Daltons) having at least one carboxyl group and at least one primary or secondary amine group capable of forming peptide bonds. The term “peptide” is intended to include any molecule comprising at least two amino acids joined by a peptide bond and therefore includes di-peptides, tri-peptides, oligopeptides, and polypeptides having up to about 20 amino acid residues. The term “peptide” also embraces structures having one or more linkers, spacers, or terminal groups which are not amino acids.

Peptides

The peptides of the invention may comprise, consist essentially of, or consist of the sequence QILSKLRL (Gln-Ile-Leu-Ser-Lys-Leu-Arg-Leu) (SEQ ID NO: 1), or any 3, 4, 5, 6, or 7 amino acid fragments thereof. Tripeptide fragments include the following sequences: QIL (Gln-Ile-Leu) (SEQ ID NO: 2), ILS (Ile-Leu-Ser) (SEQ ID NO: 3), LSK (Leu-Ser-Lys) (SEQ ID NO: 4), SKL (Ser-Lys-Leu) (SEQ ID NO: 5), KLR (Lys-Leu-Arg) (SEQ ID NO: 6), and LRL (Leu-Arg-Leu) (SEQ ID NO: 7). Tetrapeptide fragments include the following sequences: QILS (Gln-Ile-Leu-Ser) (SEQ ID NO: 8), ILSK (Ile-Leu-Ser-Lys) (SEQ ID NO: 9), LSKL (Leu-Ser-Lys-Leu) (SEQ ID NO: 10), SKLR (Ser-Lys-Leu-Arg) (SEQ ID NO: 11), and KLRL (Lys-Leu-Arg-Leu) (SEQ ID NO: 12). Pentapeptide fragments include the following sequences: QILSK (SEQ ID NO: 13), ILSKL (SEQ ID NO: 14), LSKLR (SEQ ID NO: 15), or SKLRL (SEQ ID NO: 16). A peptide comprising the sequence QILSKLRL (SEQ ID NO: 1) or derivative or fragment thereof, including SEQ ID NOs: 2-12, may have one or more additional amino acids joined to the amino and/or carboxy terminus via peptide bonds. In some embodiments, the peptide comprising the sequence QILSKLRL (SEQ ID NO: 1), or fragments thereof (SEQ ID NOs: 2-16) will have from 4 to 16 or from 4 to 12 or from 4 to 10 or from 4-9 or from 4-8 or from 4-7 or from 4-6 amino acids. In some embodiments, the peptide will comprise a hydrocarbon chain on the amino and/or carboxyl terminus, including, without limitation, C₁₋₂₄ or C₆₋₁₈ or C₁₂₋₁₈ aliphatic hydrocarbons, which may be straight chained or branched or cyclic. In some embodiments, the peptide includes the reaction product of a peptide with a fatty acid or fatty alcohol. For example, the N-terminus may be reacted with a C₆₋₂₄ fatty acid (e.g., palmitic acid) to form an amide bond. The carboxyl terminus may be reacted with a C₆₋₂₄ fatty alcohol (e.g., cetyl alcohol) to form an ester. These fatty derivatives may improve the lipophilicity of the peptide. The phrase “consisting essentially of,” as used herein, is intended to mean that additional amino acids or other residues may be present at either terminus of the peptide and/or on a side chain provided they do not substantially impair the activity of the peptide to stimulate collagen production. In one embodiment, the peptide comprises the structure of formula (I):

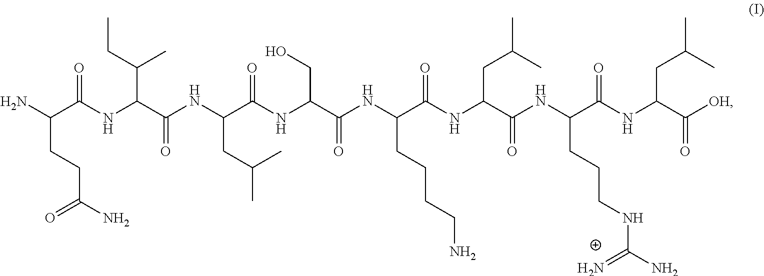 [SMILES: CCC(C)C(NC(=O)C(N)CCC(N)=O)C(=O)NC(CC(C)C)C(=O)NC(CO)C(=O)NC(CCCCN)C(=O)NC(CC(C)C)C(=O)NC(CCCNC(=N)N)C(=O)NC(CC(C)C)C(=O)O]

including zwitterions thereof.

In one embodiment, the peptide comprises only natural amino acids and includes the sequence (L)-Gln-(L)-Ile-(L)-Leu-(L)-Ser-(L)-Lys-(L)-Leu-(L)-Arg-(L)-Leu (SEQ ID NO: 1). In another embodiment, the peptide comprises non-natural amino acids, and may, for example, include the sequence (D)-Gln-(D)-Ile-(D)-Leu-(D)-Ser-(D)-Lys-(D)-Leu-(D)-Arg-(D)-Leu (SEQ ID NO: 17). In yet another embodiment the peptide comprises a combination of natural (L-) and non-natural (D-) aminoacids. For example, one or more D-amino acids may be added at the amino and/or carboxyl terminus to alter the functionality, selectivity, or hydrolytic stability of the peptide. Unless otherwise specified, the peptides referenced in the present disclosure comprise only natural (L-) amino acids.

Topically acceptable salts, esters, and prodrugs (collectively “derivatives”) of the peptides of the invention are also suitable. The esters may include C₁₋₂₄ aliphatic hydrocarbon esters of the carboxyl terminus and/or the serine side chain hydroxyl, including C₁₋₂₄ or C₁₋₁₈ or C₁₋₁₆ or C₁₋₁₂ or C₁₋₆ alkyl esters. Salts will typically be acid addition salts formed by the reaction of the peptide with an inorganic or an organic acid. Inorganic acids include mineral acids such as HCl and H₂SO₄, and the like. Organic acids include citric, benzoic, tartaric, malic, maleic, succinic, acetic, and propionic acid. Prodrugs include any esters or amides that hydrolyze in vivo to yield the peptide. Examples of suitable prodrugs can be found in the book titled “Prodrugs and Targeted Delivery: Towards Better ADME Properties,” Volume 47 (2011), published by WILEY-VCH Verlag & Co, which is herein incorporated by reference in its entirety. In one embodiment, the prodrug is formed by reacting the peptide with glyoxylic acid to produce peptidyl-α-hydroxylglycine derivatives having improved stability. In other embodiment the prodrugs may include terminal N-acetyl derivatives, side chain N-acetyl derivatives, N-hydroxy methylation or N-phthalidation of its N-terminus and/or side chain. In some embodiments either terminus may be functionalized with an amino acid of the form H₂N—(CH₂)ₙ—CO₂H where “n” is an integer from 1-10, including amino valeric acid. In some embodiments, a lysine-amino valeric acid group is added at either terminus through a peptide bond.

It is within the skill in the art to prepare the peptides of the invention using, for example, conventional protection and activation chemistry. Typically, the amino functionality of a first amino acid is protected with a removable amino protecting group and the carboxyl functionality of a second amino acid is protected with a removable carboxyl protecting group. Suitable amine protecting groups include, without limitation, benzoyloxycarbonyl (Cbz), tert-butoxycarbonyl (t-Boc), and 9-flourenylmethloxycarbonyl (FMOC). The carboxyl group may be protected by forming an acid or base labile ester such as a methyl, ethyl, benzyl, or trimethylsilyl esters. After protection, the first and second amino acids are reacted in a suitable solvent such as water or DMF in the presence of an in situ activating agent such as N,N′-dicyclohexylcarbodiimide (DCCI), diisopropylcarbodiimide (DIPCDI), or 1-ethyl-3-(3′-dimethylaminopropyl)carbodiimide (EDCI) to effect peptide bond formation. Reactive moieties on the side chains of either amino acid are protected with protecting groups such as tert-butyl or benzyl for OH and SH; methyl, ethyl, tert-butyl or benzyl for carboxyl groups, and 2,2,5,7,8-pentamethylchroman-6-sulphonyl for the —NHC(NH₂)═NH functionality of Arg. Following the coupling reaction, selective deprotection of the amino group of the first amino acid is accomplished by acid hydrolysis under conditions that do not remove the carboxyl protecting group of the second amino acid. The procedure is repeated with additional amino protected amino acids. Solid phase synthesis, such as the well-known Merrifield method, is especially useful for synthesizing the peptides of the invention. Lysine-amino valeric acid (K-ava) derivatives are described in U.S. Pat. No. 8,551,956, the disclosure of which is hereby incorporated by reference.

Topical Compositions

The compositions according to the invention may be formulated in a variety of forms for topical application and will typically comprise from about 0.000001% by weight to about 20% by weight of the peptide. More typically, the peptide will comprise from about 0.00001% by weight to about 10% by weight, and more preferably from about 0.00001% by weight to about 5% by weight of the composition. In one embodiment, the active peptide or a fragment or derivative thereof will comprise from about 0.00001% by weight to about 0.0001% by weight or to about 0.001% by weight or to about 0.1% by weight of the composition. The compositions may comprise an effective amount of the peptide, by which is meant an amount sufficient to stimulate production of collagen in the skin. In other embodiments, the amount of peptide will be sufficient to diminish the appearance of dermatological signs of aging in a given area of skin when topically applied thereto daily for a period of at least eight weeks.

The peptides of the invention are provided in physiologically acceptable vehicles or carriers. The vehicle may be either hydrophobic or hydrophilic. Suitable, hydrophobic carriers include, for example, waxy non-ionic substances commonly used in cosmetics, such as esters and ethers of fatty alcohols and of fatty acids, with carbon chain length from C₄ to C₂₂, preferably from C₈ to C₁₈, or from C₁₂ to C₁₈.

Examples of a fatty hydrophobic carriers include isopropyl myristate, isopropyl palmitate, octyl palmitate, isopropyl lanolate, acetylated lanolin alcohol, the benzoate of C₁₂-C₁₅ alcohols, cetearyl octanoate, cetyl palmitate, myristyl myristate, myristyl lactate, cetyl acetate, propylene glycol dicaprylate/caprate, decyl oleate, acetylated lanolin, stearyl heptanoate, diisostearyl malate, octyl hydroxystearate, octyl hydroxystearate, isopropyl isostearate, and the like.

Suitable hydrophilic carriers may comprise, for example, water, lower alcohols (C₁₋₆), glycols and alkoxylated glycols commonly used in cosmetics, including ethylene glycol, diethylene glycol, triethylene glycol, propylene glycol, dipropylene glycol, and the like.

The topically acceptable vehicle may be in the form of an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. As used herein, the term “oil” includes silicone oils unless otherwise indicated. The emulsion may include an emulsifier, such as a nonionic, anionic or amphoteric surfactant, or a gellant, typically in an amount from about 0.001% to about 5% by weight.

The topically acceptable vehicle may include water; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane (IDD) and isohexadecane; silicone oils such as cyclomethicone, dimethicone, dimethicone cross-polymer, polysiloxanes and their derivatives, preferably organomodified derivatives including PDMS, dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous vehicles may include one or more solvents miscible with water, including lower alcohols, such as ethanol, isopropanol, and the like. The vehicle may comprise from about 50% to about 99% by weight of the composition.

In one embodiment of the invention, the compositions may include one or more additional skin actives, including but not limited to, retinoids, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, and advanced glycation end-product (AGE) inhibitors, to name but a few. The amounts of these various ingredients are those conventionally used in the cosmetic field to achieve their intended purpose, and range individually or collectively typically from about 0.001 wt % to about 20 wt % by weight of the composition. The nature of these ingredients and their amounts must be compatible with the production and function of the compositions of the disclosure.

Exemplary anti-aging components include, without limitation, botanicals (e.g., Butea frondosa extract, Tiliacora triandra extract, Portulaca oleracea, Melicope elleryana, etc.); phytol; phytonic acid; retinoids; hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof, etc.), to name a few.

Exemplary retinoids include, without limitation, retinoic acid (e.g., all-trans, or 9-cis, or 13-cis), and derivatives thereof, retinaldehyde, retinol (Vitamin A) and esters thereof, such as retinyl palmitate, retinyl acetate and retinyl propionate, and salts thereof. Particular mention may be made of retinol. When present, the retinoids will typically be included in amounts from about 0.0001% to about 5% by weight, more typically from about 0.01% to about 2.5% by weight, or from about 0.1% to about 1.0% by weight. Compositions according to this embodiment will typically include an antioxidant such as ascorbic acid and/or BHT and/or a chelating agent such as EDTA or a salt thereof (e.g., disodium EDTA) in amounts effective to stabilize the retinoid (e.g., 0.0001%-5%).

In another embodiment, the topical compositions of the present invention may also include one or more of the following: a skin penetration enhancer; an emollient, such as isopropyl myristate, petrolatum, volatile or non-volatile silicones oils (e.g., methicone, dimethicone), ester oils, mineral oils, and fatty acid esters; a humectant, such as glycerin, hexylene glycol or caprylyl glycol; a skin plumper, such as palmitoyl oligopeptide, collagen, collagen and/or glycosaminoglycan (GAG) enhancing agents; a sunscreen, such as avobenzone or octyl methoxycinnamate; an exfoliating agent; and an antioxidant.

Suitable exfoliating agents include, for example, alpha-hydroxy acids, beta-hydroxy acids, oxa-acids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. One exemplary exfoliating agent is glycolic acid. When present, the exfoliating agent may comprise from about 0.001% to about 20% by weight of the composition.

Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives, including tocopheryl acetate; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Antioxidants may comprise, individually or collectively, from about 0.001% to about 10% (w/w), or from about 0.01% to about 5% (w/w) of the total weight of the composition.

Other additives include: vitamins, such as tocopherol and ascorbic acid; vitamin derivatives such as ascorbyl monopalmitate, tocopheryl acetate, and Vitamin E palmitate; thickeners such as hydroxyalkyl cellulose, carboxymethylcellulose, carbombers, and vegetable gums such as xanthan gum; gelling agents, such as ester-terminated polyester amides; structuring agents; metal chelating agents such as EDTA or salts thereof; pigments; colorants; and pH adjusters (citric acid, ethanolamine, sodium hydroxide, etc.). The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. The foregoing may individually or collectively comprise from about 0.0001% to about 20% by weight of the composition.

In addition, the compositions contemplated by this disclosure can include one or more compatible cosmetically acceptable adjuvants commonly used and known by the skilled practitioner, such as colorants, pearls, chromalites, micas, pigments, dyes, fragrances, emollients, humectants, preservatives, vitamins, chelators, thickeners, anesthetics, anti-allergenics, antifungals, antimicrobials, other anti-inflammatory agents, antioxidants, antiseptics, depigmenting agents, film formers, insect repellents, pharmaceutical agents, photostabilizing agents, sunscreens, stabilizers, surfactants, thickeners, viscosity modifiers, and botanicals. The topical compositions of the present disclosure may also include a skin penetration enhancer, a surface smoother, a skin plumper, an optical diffuser, an exfoliation promoter, and an antioxidant. Details with respect to these and other suitable cosmetic ingredients can be found in the “International Cosmetic Ingredient Dictionary and Handbook,” 10th Edition (2004), published by the Cosmetic, Toiletry, and Fragrance Association (CTFA), at pp. 2177-2299, which is herein incorporated by reference in its entirety. The amounts of these various substances are those that are conventionally used in the cosmetic or pharmaceutical fields, for example, they can constitute from about 0.01% to about 20% of the total weight of the composition.

A sunscreen may be included to protect the skin from damaging ultraviolet rays. In an illustrative embodiment of the present disclosure, the sunscreen provides both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

In one embodiment, the topical composition will have a pH range from 1 to 13, with a pH in the range of from 2 to 12 being typical. In some embodiment, the composition will have a pH in the range of from 3.5 to 7 or from 7-10.5. In some embodiments, the pH will be in the range of 3-4, or 4-5, or 5-6, or 6-7, or 7-8, or 8-9, or 9-10, or 10-11, or 11-12. Suitable pH adjusters such as sodium hydroxide, citric acid and triethanolamine may be added to bring the pH within the desired range.

Another embodiment of the present disclosure is directed to the delivery of the described compositions by the use of targeted delivery systems, for example, liposomes, microspheres (see, e.g., U.S. Pat. No. 5,770,222 to Unger et al.), and the like, so that the components and/or active constituents can more readily reach and affect the subcutaneous layer of the area of application, e.g., face or neck, or the other area of the skin.

The compositions may be formulated in a variety of product forms, such as, for example, a lotion, cream, serum, spray, aerosol, cake, ointment, essence, gel, paste, patch, pencil, towelette, mask, stick, foam, elixir, concentrate, and the like, particularly for topical administration. Preferably the composition is formulated as a lotion, cream, ointment, or gel.

The invention also provides a method for ameliorating and/or preventing signs of human skin photo- and intrinsic aging comprising topically applying the compositions of the invention. The compositions of the invention are preferably applied to affected skin areas once or twice daily for as long as is necessary to achieve desired anti-aging results.

Methods of Treatment

Methods are provided for enhancing the production of collagen in human skin comprising topically applying to an area of the skin in need thereof (e.g., sagging skin, thinning skin, skin suffering from wrinkles and fine lines, etc.) a topical composition comprising a topically acceptable vehicle, and an effective amount of a peptide of the invention (e.g., SEQ ID NOs: 1-16), for a time sufficient to improve the appearance thereof. The treatment may be at least once or twice daily and may last for a period of at least four weeks, typically at least eight weeks or longer. The composition may optionally further comprise a retinoid and/or an alpha-hydroxy acid (e.g., glycolic acid) and/or a beta-hydroxy acid (e.g., salicylic acid or a derivative) in amounts effective to improve the appearance of skin.

In another aspect of the invention, the compositions are applied topically to improve the aesthetic appearance of human skin. The method comprises topically applying to an area of the skin in need thereof a composition comprising an effective amount of a peptide of the invention (e.g., SEQ ID NOs: 1-16) for a time sufficient to improve the aesthetic appearance of said human skin. The compositions are topically applied to the skin in effective amounts, by which is meant an amount sufficient to achieve a measurable improvement in skin health or reduction in one or more dermatological signs of aging with daily (once, twice, etc.) administration, typically for a period of at least one week or more.

The aesthetic improvement of human skin may be an improvement of any attribute or characteristic of skin, including without limitation:

(a) treatment, reduction, and/or prevention of fine lines or wrinkles;

(b) reduction of skin pore size;

(c) improvement in skin thickness, plumpness, and/or tautness;

(d) improvement in skin smoothness, suppleness and/or softness;

(e) improvement in skin tone, radiance, and/or clarity;

(f) improvement in procollagen, and/or collagen production;

(g) improvement in maintenance and remodeling of elastin;

(h) improvement in skin texture and/or promotion of retexturization;

(i) improvement in skin barrier repair and/or function;

(j) improvement in appearance of skin contours;

(k) restoration of skin luster and/or brightness;

(l) replenishment of essential nutrients and/or constituents in the skin;

(m) improvement of skin appearance decreased by aging and/or menopause;

(n) improvement in skin moisturization;

(o) increase in skin elasticity and/or resiliency;

(p) treatment, reduction, and/or prevention of skin sagging;

(q) improvement in skin firmness; and

(r) reduction of pigment spots and/or mottled skin; and

(s) improvement of optical properties of skin by light diffraction or reflection.

In a related implementation, a method is provided for the treatment of wrinkles and/or fine lines on the skin human skin (typically, skin of the face) comprising topically applying to an area of the skin in need thereof (e.g., applying to a wrinkle or fine line) a composition comprising a peptide of the invention (e.g., SEQ ID NOs: 1-16), for a time sufficient to improve the aesthetic appearance of said human skin. The treatment may be a least once or twice daily and may last for a period of at least four weeks, typically at least eight weeks or longer. The composition may optionally further comprise a retinoid (e.g., retinol or retinol palmitate) and/or an alpha-hydroxy acid (e.g., glycolic acid) and/or a beta-hydroxy acid (e.g., salicylic acid or derivative) in amounts effective to improve the appearance of skin.

In yet another aspect of the invention, methods are provided for reducing the severity of, reducing the number of, or preventing or forestalling the onset of, wrinkles or fine lines on human skin comprising topically applying to an area of the skin in need thereof (e.g., wrinkled skin), an effective amount (e.g., 0.000001%-1% by weight, w/w) of a peptide of the invention (e.g., SEQ ID NOs: 1-16) in combination with an effective amount (e.g., 0.0001%-5% by weight, w/w) of retinol and/or an effective amount (e.g., 0.00001%-5% by weight, w/w) of an alpha-hydroxy acid (e.g., glycolic acid) and/or a beta-hydroxy acid (e.g., salicylic acid).

The invention provides a method for treating aging skin by topically applying a composition comprising a collagen-stimulating peptide (e.g., SEQ ID NOs: 1-16), typically in a physiologically acceptable vehicle, over the affected area for a period of time sufficient to remediate, reverse, reduce, ameliorate, or prevent dermatological signs of aging. Generally, the improvement in the condition and/or aesthetic appearance is selected from the group consisting of: reducing dermatological signs of chronological aging, photo-aging, hormonal aging, and/or actinic aging; preventing and/or reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; improving the appearance of suborbital lines and/or periorbital lines; reducing the appearance of crow's feet; rejuvenating and/or revitalizing skin, particularly aging skin; reducing skin fragility; preventing and/or reversing of loss of glycosaminoglycans and/or collagen; ameliorating the effects of estrogen imbalance; preventing skin atrophy; preventing, reducing, and/or treating hyperpigmentation or hypopigmentation; minimizing skin discoloration; improving skin tone, radiance, clarity and/or tautness; preventing, reducing, and/or ameliorating skin sagging; improving skin firmness, plumpness, suppleness and/or softness; improving procollagen and/or collagen production; improving skin texture and/or promoting retexturization; improving skin barrier repair and/or function; improving the appearance of skin contours; restoring skin luster and/or brightness; minimizing dermatological signs of fatigue and/or stress; resisting environmental stress; replenishing ingredients in the skin decreased by aging and/or menopause; improving communication among skin cells; increasing cell proliferation and/or multiplication; increasing skin cell metabolism decreased by aging and/or menopause; retarding cellular aging; improving skin moisturization; enhancing skin thickness; slowing or halting skin thinning; increasing skin elasticity and/or resiliency; enhancing exfoliation; improving microcirculation; decreasing and/or preventing cellulite formation; and any combinations thereof. In some embodiments, each of the forgoing is associated with female skin.

In some embodiments, the peptides of the invention (e.g., SEQ ID NOs: 1-16) will be used to reduce the severity of fine lines or wrinkles, often in combination with retinol. The composition will typically be applied to the skin one, two, or three times daily for as long as is necessary to achieve desired results. The treatment regimen may comprise daily application for at least one week, at least two weeks, at least four weeks, at least eight weeks, or at least twelve weeks or more. Chronic treatment regimens are also contemplated. The effect of a composition on the formation or appearance of fine lines and wrinkles can be evaluated qualitatively, e.g., by visual inspection, or quantitatively, e.g., by microscopic or computer assisted measurements of wrinkle morphology (e.g., the number, depth, length, area, volume and/or width of wrinkles per unit area of skin). In one embodiment, the compositions of the invention will be applied to the skin in an amount from about 0.001 to about 100 mg/cm², more typically from about 0.01 to about 20 mg/cm², or from about 0.1 to about 10 mg/cm².

It is also contemplated that the compositions of the invention will be useful for treating thin skin by topically applying the composition comprising the active peptides (e.g., SEQ ID NOs: 1-16) to thin skin of an individual in need thereof. “Thin skin” is intended to include skin that is thinned due to chronological aging, menopause, or photo-damage and skin that is thinning prematurely. In some embodiments, the treatment is for thin skin in men, whereas other embodiments treat thin skin in women, pre-menopausal or post-menopausal, as it is believed that skin thins differently with age in men and women, and in particular in women at different stages of life.

The method of the invention may be employed prophylactically to forestall aging including in individuals that have not manifested signs of skin aging, most commonly in individuals under 25 years of age. The method may also reverse or treat signs of aging once manifested as is common in individuals over 25 years of age, or to slow the progression of dermatological aging in such individuals.

In one embodiment, the compositions of the invention comprising active peptides (e.g., SEQ ID NOs: 1-16) are applied to human skin to reduce sebum production or improve the appearance of skin affected by cellulite, and/or reduce unwanted lipogenesis or increase lipolysis. In this embodiment, the peptides of the invention can be formulated in topically acceptable vehicles (as described herein) and may include one or more additional agents such as anti-acne ingredients (e.g., salicylic acid, benzoyl peroxide and other peroxides, sulfur, retinoids, etc.) in the case of a facial composition, or, in the case of a cellulite treatment, the formulation may comprise any ingredients suitable for treatment of cellulite, including without limitation, perilla oil and other unsaturated fatty oils and omega-3 fatty acids such as alpha-linolenic acid; caffeine; theophylline; xanthines; retinoids (e.g., retinol); and the like. A cellulite treatment according to the invention will typically be applied topically to skin suffering from cellulite, including skin of the buttocks and thighs for a period of time sufficient to improve the appearance thereof, including for example, daily treatment for at least four weeks, at least eight weeks, at least twelve weeks, or longer. In one embodiment, the compositions are topically applied to treat acne.

In certain embodiments, the compositions described herein comprising active peptides (e.g., SEQ ID NOs: 1-16) can be used to treat and/or prevent hyper-pigmentation of skin and/or of the hair, for example, to lighten skin or hair. In some embodiments, the compositions are topically applied to the skin or hair, for example to an area of hyper-pigmented skin or hair. Hyper-pigmentation includes any coloration of an individual's skin or hair that is darker than desired by the individual and that is caused by melanocytes. Such unwanted pigmentation may also be called discoloration. Hyper-pigmented areas of the skin include areas of discrete or mottled hyper-pigmentation. Areas of discrete hyper-pigmentation can be distinct, uniform areas of darker color and may appear as brown spots or freckles on the skin, including marks commonly called pigment spots or “age spots.” Areas of mottled hyper-pigmentation of the skin can be dark blotches that are larger and more irregular in size and shape than areas of discrete pigmentation. Areas of hyper-pigmentation also include areas of tanned skin, for example, skin tanned due to UV exposure. Hyper-pigmented hair includes any shade of hair that is darker than desired.

Treating hyper-pigmentation or hyper-pigmented skin/hair refers to eradicating, reducing, ameliorating, or reversing one or more of the unwanted features associated with hyper-pigmentation, such as producing a perceptible lightening of the skin or hair in the affected area. Lightening hyper-pigmented areas of the skin may be desirable, in one embodiment, in diminishing age spots; lightening a suntan; evening or optimizing skin tones, e.g., in areas of mottled hyper-pigmentation; in treating melasmic and chloasmic patches, freckles, after-burn scars, and post-injury hyper-pigmentation. Preventing hyper-pigmentation or hyper-pigmented skin refers to affording skin, not yet affected by hyper-pigmentation, a benefit that serves to avoid, delay, forestall, or minimize one or more unwanted features associated with skin hyper-pigmentation, such as reducing the darkness or size of hyper-pigmented areas that eventually develop.

In another embodiment, the peptides of the invention (e.g., SEQ ID NOs: 1-16) are intended for oral use, including for pharmaceutical use. Pharmaceutical formulations will include pharmaceutically acceptable carriers (i.e., diluents and excipients). The pharmaceutical compositions may be included in solid dosage forms, including compressed tablets and capsules, or in liquid or powder forms (including lyophilized powders of the peptide suitable for reconstitution with water). Pharmaceutical dosage forms will typically include from about 0.1 mg to about 200 mg, or from about 1 mg to about 100 mg of the peptides of the invention. The dosage forms may be immediate release, in which case they will typically comprise a water-soluble or dispersible carrier such as microcrystalline cellulose, mannitol, hydroxypropyl methyl cellulose, PVP or the like, or may be delayed, sustained, or modified release, in which case they may comprise water-insoluble polymers such as cellulose ethers (e.g., ethylcellulose), alone or in combination with water soluble or dispersible polymers, to regulate the rate of dissolution of the dosage form in the stomach.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### EXAMPLES

The following example illustrates a specific aspect of the instant description. The example should not be construed as limiting, as the example merely provides specific understanding and practice of the embodiments and its various aspects.

### Example 1

The peptide of Formula I (QILSKLRL (SEQ ID NO: 1)) was synthesized by GenScript (Piscataway, N.J.).

Human dermal fibroblast cells were grown in a 96 well plate in DMEM media (available from Corning, N.Y.) supplemented with 10% Fetal Bovine Serum (FBS) and L-glutamine (0.07×10⁵ cells/plate). After reaching about 75% confluence, cells were transferred into DMEM media without FBS and incubated for 4-6 hours. Next, cells were treated with peptide of Formula I at 0.00001%, 0.0001%, 0.001% final concentration in DMEM media without FBS for 48 h. After treatment the media were collected, and cell viability was measured using MTT. The amount of secreted collagen was tested in the media using HTRF human pro-collagen I kit (Cisbio Inc, Bedford, Mass.).

Results are summarized in Table 1 below as percent change of pro-collagen I production relative to vehicle control:

TABLE 1

| | | Increase in Pro-Collagen I |
|---|---|---|
| Peptide Sequence | Peptide Concentration | Production |
| QILSKLRL (SEQ ID NO: 1) | 0.001% | +10-30% |

As shown in Table 1, a peptide of Formula I effectively increases pro-collagen I production in human dermal fibroblast cells by 10-30% at 0.001% concentration.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

1. A method for improving the health and/or appearance of human skin affected by dermatological signs of aging comprising topically applying to an area of skin in need thereof a composition comprising, in a topically acceptable vehicle, an effective amount of a peptide comprising the sequence QILSKLRL (SEQ ID NO: 1), QIL (SEQ ID NO: 2), ILS (SEQ ID NO: 3), LSK (SEQ ID NO: 4), SKL (SEQ ID NO: 5), KLR (SEQ ID NO: 6), LRL (SEQ ID NO: 7), QILS (SEQ ID NO: 8), ILSK (SEQ ID NO: 9), LSKL (SEQ ID NO: 10), SKLR (SEQ ID NO: 11), KLRL (SEQ ID NO: 12), QILSK (SEQ ID NO: 13), ILSKL (SEQ ID NO: 14), LSKLR (SEQ ID NO: 15), or SKLRL (SEQ ID NO: 16), for a time sufficient to improve the appearance and/or health of said skin.

2. The method according to claim 1, wherein said peptide increases collagen production in skin.

3. The method according to claim 1, wherein said improvement in the appearance of skin affected by dermatological signs of aging is selected from a group consisting of:
(a) treatment, reduction, and/or prevention of fine lines and/or wrinkles;
(b) reduction of skin pore size;
(c) improvement in skin thickness, plumpness, and/or tautness;
(d) improvement in skin smoothness, suppleness and/or softness;
(e) improvement in skin tone, radiance, and/or clarity;
(f) improvement in procollagen, and/or collagen production;
(g) improvement in maintenance and remodeling of elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) improvement of skin appearance decreased by aging and/or menopause;
(n) improvement in skin moisturization;
(o) increase in skin elasticity and/or resiliency;
(p) treatment, reduction, and/or prevention of skin sagging;
(q) improvement in skin firmness; and
(r) reduction of pigment spots and/or mottled skin; and
(s) improvement of optical properties of skin by light diffraction or reflection.

4. The method according to claim 1, wherein said improvement in the appearance of skin affected by dermatological signs of aging includes reduction in the fine lines and/or wrinkles.

5. The method according to claim 1, wherein said improvement in the appearance of skin affected by dermatological signs of aging includes reduction in skin sagging.

6. The method according to claim 1, wherein said improvement in the appearance of skin affected by dermatological signs of aging includes thickening of said skin.

7. The method according to claim 1, wherein said composition is applied at least once daily.

8. The method according to claim 1, wherein said composition is applied at least once daily for at least eight weeks.

9. The method according to claim 1, wherein said topically acceptable vehicle comprises a water-in-oil, oil-in-water, silicone-in-water, or water-in-silicone emulsion, and further comprises an emulsifier.

10. The method according to claim 1, wherein said effective amount is from about 0.000001% to about 5% by weight.

11. The method according to claim 1, wherein said area of skin comprises skin of the face.

12. The method according to claim 1, wherein said composition comprises an additional active ingredient selected from the group consisting of alpha-hydroxy acids, glycolic acid, thodipropionic acid or esters thereof, salicylic acid, N-acetyl tyrosinamide, phytol, and retinoids.

13. The method according to claim 12, wherein said composition comprises retinol.

14. A composition comprising an effective amount of a peptide comprising the sequence QILSKLRL (SEQ ID NO: 1), QIL (SEQ ID NO: 2), ILS (SEQ ID NO: 3), LSK (SEQ ID NO: 4), SKL (SEQ ID NO: 5), KLR (SEQ ID NO: 6), LRL (SEQ ID NO: 7), QILS (SEQ ID NO: 8), ILSK (SEQ ID NO: 9), LSKL (SEQ ID NO: 10), SKLR (SEQ ID NO: 11), KLRL (SEQ ID NO: 12), QILSK (SEQ ID NO: 13), ILSKL (SEQ ID NO: 14), LSKLR (SEQ ID NO: 15), or SKLRL (SEQ ID NO: 16) and a topically acceptable vehicle.

15. The composition of claim 14, wherein said effective amount is from about 0.00001% to about 5% by weight.

16. The composition of claim 14, wherein said effective amount is from about 0.00001% to about 0.001% by weight.

17. The composition of claim 14, wherein said topically acceptable vehicle comprises a water-in-oil, oil-in-water, silicone-in-water, or water-in-silicone emulsion and further comprises an emulsifier.

18. The composition of claim 14 further comprising an additional active ingredient selected from the group consisting of alpha-hydroxy acids, thiodipropionic acid or esters thereof, salicylic acid, niacinamide, hexyl resorcinol, phytol, and retinoids.

19. The composition of claim 18 further comprising retinol.

20. The composition of claim 14 further comprising at least one other ingredient selected from the group consisting of a film forming polymer, a thickener, a pH adjuster, a preservative, an emulsifier, a gelling agent, an antioxidant, an emollient, a humectant, a fragrance, and a colorant.
